(19) United States
(12) Patent Application Publication
(10) Pub. No.: US 2025/0075059 A1
(43) Pub. Date: Mar. 6, 2025

(54) SILICONE INFUSED WITH SILVER

(71) Applicant: Thomas E. Garrison, Scottsdale, AZ (US)

(72) Inventor: Thomas E. Garrison, Scottsdale, AZ (US)

(21) Appl. No.: 18/457,668

(22) Filed: Aug. 29, 2023

(51) Int. Cl. C08K 7/18; A61K 9/70; A61K 33/38; A61K 47/34

(52) U.S. Cl. CPC C08K 7/18; A61K 9/7015; A61K 33/38; A61K 47/34; C08K 2201/005; C08K 2201/011

(57) ABSTRACT

Apparatus, mixtures, and methods including silicone infused with silver particles are disclosed herein. One apparatus includes a silicone strip including silicone infused with silver particles. A mixture includes silicone infused with silver particles. A method includes identifying a target surface and applying a mixture to the target surface, in which the mixture includes silicone and an amount of silver particles infused in the silicone.

### FIELD

The subject matter disclosed herein relates to silicone and, more particularly, relates to silicone infused with silver.

### BACKGROUND

Silicone has several uses in medicine. While effective for some uses, silicone by itself is not as effective as it could be for at least some uses including, for example, treating scars and/or scar contractures.

### BRIEF SUMMARY

The subject matter of the present disclosure provides examples of apparatus and mixtures that include silicone infused with silver that can increase the healing properties of silicone. Accordingly, the subject matter of the present application has been developed in response to the present state of the art, and in particular, in response to the limited efficacy of conventional silicone and/or the limited efficacy of conventional apparatus and methods that use silicone for treating scars and/or scar contractures.

Disclosed herein is an apparatus including silicone forming a silicone strip and an amount of silver (Ag) particles infused in the silicone strip. The preceding subject matter of this paragraph characterizes example 1 of the present disclosure.

The Ag particles in the mixture include nano-silver particles. The preceding subject matter of this paragraph characterizes example 2 of the present disclosure, wherein example 2 also includes the subject matter according to example 1, above.

The mixture includes 0.5 ppm to 100 ppm by weight of the Ag particles. The preceding subject matter of this paragraph characterizes example 3 of the present disclosure, wherein example 3 also includes the subject matter according to any one of examples 1 or 2, above.

The mixture includes 10 ppm by weight of the Ag particles. The preceding subject matter of this paragraph characterizes example 4 of the present disclosure, wherein example 4 also includes the subject matter according to any one of examples 1 or 2, above.

The mixture includes 32 ppm by weight of the Ag particles. The preceding subject matter of this paragraph characterizes example 5 of the present disclosure, wherein example 5 also includes the subject matter according to any one of examples 1 or 2, above.

The nano-silver particles include a size less than or equal to one micrometer (1 μm). The preceding subject matter of this paragraph characterizes example 6 of the present disclosure, wherein example 6 also includes the subject matter according to any one of examples 1 or 2, above.

The Ag particles in the mixture include colloidal silver particles. The preceding subject matter of this paragraph characterizes example 7 of the present disclosure, wherein example 7 also includes the subject matter according to example 1, above.

Also disclosed herein is a mixture including silicone and an amount of silver (Ag) particles infused in the silicone in which the mixture includes one of a liquid or a gel. The preceding subject matter of this paragraph characterizes example 8 of the present disclosure.

The Ag particles in the mixture include nano-silver particles. The preceding subject matter of this paragraph characterizes example 9 of the present disclosure, wherein example 9 also includes the subject matter according to example 8, above.

The mixture includes 0.5 ppm to 100 ppm by weight of the Ag particles. The preceding subject matter of this paragraph characterizes example 10 of the present disclosure, wherein example 10 also includes the subject matter according to any one of examples 8 or 9, above.

The mixture includes 10 ppm by weight of the Ag particles. The preceding subject matter of this paragraph characterizes example 11 of the present disclosure, wherein example 11 also includes the subject matter according to any one of examples 8 or 9, above.

The mixture includes 32 ppm by weight of the Ag particles. The preceding subject matter of this paragraph characterizes example 12 of the present disclosure, wherein example 12 also includes the subject matter according to any one of examples 8 or 9, above.

The nano-silver particles include a size less than or equal to one micrometer (1 μm). The preceding subject matter of this paragraph characterizes example 13 of the present disclosure, wherein example 13 also includes the subject matter according to any one of examples 8 or 9, above.

The Ag particles in the mixture include colloidal silver particles. The preceding subject matter of this paragraph characterizes example 14 of the present disclosure, wherein example 14 also includes the subject matter according to example 8, above.

Further disclosed herein is a method including identifying a target surface and applying a mixture to the target surface in which the mixture includes silicone and an amount of silver (Ag) particles infused in the silicone. The preceding subject matter of this paragraph characterizes example 15 of the present disclosure.

The mixture forms a silicone strip infused with the amount of the silver particles and the target surface includes skin. The preceding subject matter of this paragraph characterizes example 16 of the present disclosure, wherein example 16 also includes the subject matter according to example 15, above.

The Ag particles in the mixture include nano-silver particles. The preceding subject matter of this paragraph characterizes example 17 of the present disclosure, wherein example 17 also includes the subject matter according to example 15, above.

The mixture includes 0.5 ppm to 100 ppm by weight of the Ag particles. The preceding subject matter of this paragraph characterizes example 18 of the present disclosure, wherein example 18 also includes the subject matter according to any one of examples 15 or 17, above.

The nano-silver particles include a size less than or equal to one micrometer (1 μm). The preceding subject matter of this paragraph characterizes example 19 of the present disclosure, wherein example 19 also includes the subject matter according to any one of examples 15 or 17, above.

The Ag particles in the mixture include colloidal silver particles. The preceding subject matter of this paragraph characterizes example 20 of the present disclosure, wherein example 20 also includes the subject matter according to example 15, above.

### BRIEF DESCRIPTION OF THE DRAWINGS

A more particular description of the embodiments briefly described above will be rendered by reference to specific embodiments that are illustrated in the appended drawings. Understanding that these drawings depict only some embodiments and are not therefore to be considered to be limiting of scope, the embodiments will be described and explained with additional specificity and detail through the use of the accompanying drawings, in which:

FIG. 1 is a schematic diagram of one embodiment of a mixture including silicone and silver particles;

FIG. 2 is a schematic diagram of one embodiment of a silver particle included in the mixture of FIG. 1;

FIG. 3 is a schematic diagram of one embodiment of a molecule of coating for the silver particle illustrated in FIG. 2;

FIG. 4A is a schematic diagram of one embodiment of a silicone strip infused with silver particles;

FIG. 4B is a schematic diagram of one embodiment of a mixture including silicone infused with silver particles forming one embodiment of a silicone liquid or one embodiment of a silicone gel;

FIG. 5 is a schematic flow diagram of one embodiment of a method for generating a mixture of silicone infused with silver particles;

FIG. 6 is a schematic flow diagram of one embodiment of a method for generating a silicone strip infused with silver particles;

FIG. 7 is a schematic flow diagram of one embodiment of a method for generating a silicone tape infused with silver particles;

FIG. 8 is a schematic flow diagram of one embodiment of a method for generating a silicone gel infused with silver particles;

FIG. 9 is a schematic flow diagram of one embodiment of a method for generating a silicone liquid infused with silver particles;

FIG. 10 is a schematic flow diagram of one embodiment of a method for treating a target surface with a mixture of silicone infused with silver particles;

FIG. 11 is a schematic flow diagram of one embodiment of a method for treating a scar and/or with a silicone strip or silicone tape infused with silver particles; and

FIG. 12 is a schematic flow diagram of one embodiment of a method for treating a scar and/or with a silicone gel or silicone liquid infused with silver particles.

### DETAILED DESCRIPTION

The various embodiments disclosed herein provide apparatus, mixtures, and methods that include silicone infused with silver, which can increase the healing properties of silicone. Accordingly, the subject matter of the present application has been developed in response to the present state of the art, and in particular, in response to the limited efficacy of conventional silicone and/or the limited efficacy of conventional apparatus and methods that use silicone for treating scars and/or scar contractures.

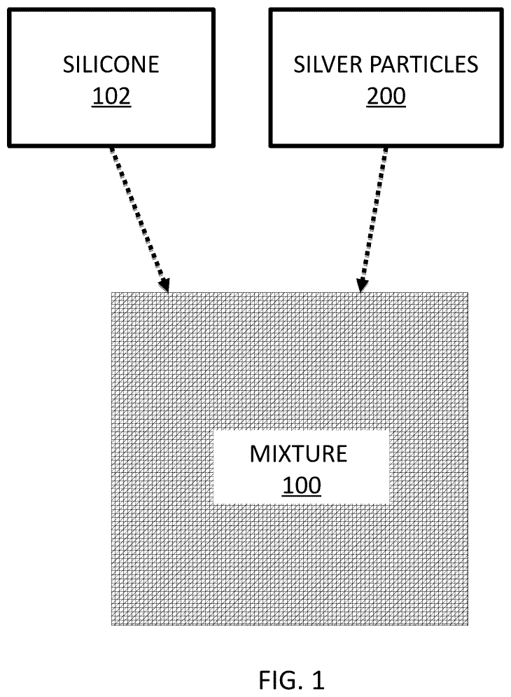

With reference to the drawings, FIG. 1 is a block diagram of one embodiment of a mixture 100 including silicone 102 and silver particles 200. In various embodiments, the silver particles 200 (e.g., a plurality of silver particles 200) are infused with, mixed with, embedded into, added to, and/or otherwise combined with the silicone 102 to form the mixture 100. As used herein, the terms silicone 102 infused with silver particles 200 and/or infused with silver can include, but are not limited to, an amount of silver particles 200 infused with, mixed with, embedded within, added to, and/or otherwise combined with an amount of silicone 102.

A mixture 100 may include any suitable amount (e.g., by weight and/or by volume) of the silicone 102 that is known or developed in the future for a particular application/use of the mixture 100 and/or application/use of silicone. Further, the mixture 100 may include any suitable amount (e.g., by weight and/or by volume) of the silver particles 200 that is known or developed in the future for a particular application/use of the mixture 100 and/or application/use of silicone.

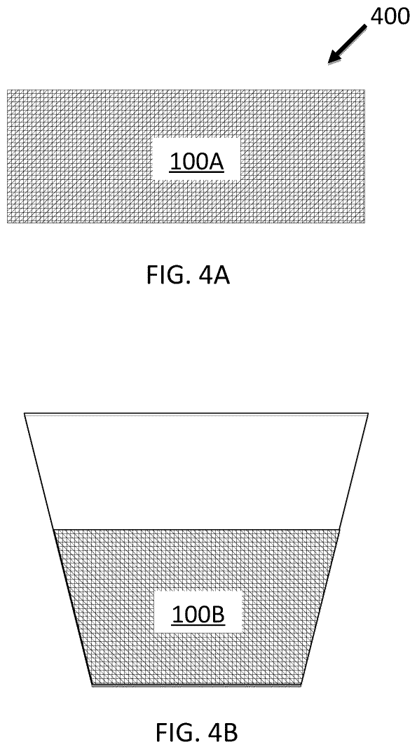

The silicone 102 may include any suitable silicone and/or type of silicone that is known or developed in the future. In addition, the silicone 102 may include any suitable compound and/or suitable mixture that includes an amount of silicone that is known or developed in the future. The silicone 102, in certain embodiments, includes medical grade silicone (see, e.g., silicone strip 400 in FIG. 4A).

In various embodiments, the silicone 102 includes a polymer including molecules that include chains of alternating silicon (Si) atoms and oxygen (O) atoms. In certain embodiments, the silicone 102 (and/or the mixture 100) may include a consistency and/or texture similar to rubber (see, e.g., silicone strip 400 in FIG. 4A).

The silicone 102, in some embodiments, includes one or more maximum members of the class II organic groups, such as methyl(CH₃) and vinyl(CH₂) linked to every silicon atom. In alternative embodiments, the silicone 102 includes an inorganic silicon-oxygen backbone chain (. . . —Si—O—Si—O—Si—O . . . ) in which two organic groups are attached to each Si center, which may be referred to as, siloxane, a polymerized siloxane, or a polysiloxane. In various embodiments, the silicone 102 may be represented by the general formula, (R₂SiO)x, in which R belongs to any one of the organic groups.

In various embodiments, the silicone 102 includes Poly-dimethylsiloxane. Here, the first substance includes metallic silicon obtained from silica sand. The metallic silicon reacts with methyl chloride (Ch3Cl) and forms dimethyldichlorosilane ([CH₃]₂Si[Cl]₂) over a copper catalyst. When this compound is made to react with water (H₂O), the atoms of chlorine get replaced by hydroxyl groups(OH). The resulting compound, silanol ([CH₃]₂Si[OH]₂), polymerizes in a condensation reaction. Every individual unit of molecules links together to form poly-dimethylsiloxane with associated loss of water.

The silver particles 200 can include any suitable particle of silver and/or type of particle of silver that is known or developed in the future. Examples of the silver particles 200 include, but are not limited to, nano-silver particles, colloidal silver particles, ionic silver particles, solution silver particles, and/or suspension silver particles, among other particles of silver and/or types of particles of silver that are possible, each of which is contemplated herein.

In various embodiments, the total amount of silver particles 200 in a mixture 100 is in the range of about 0.5 parts per million (0.5 ppm) to about 100 parts per million (100 ppm) by weight and/or volume, among other quantities of silver particles 200 that are less than about 0.5 ppm and greater than about 100 ppm by weight and/or volume of particles of silver that are possible, each of which is contemplated herein. In some embodiments, the total amount of silver particles 200 in a mixture 100 is about ten parts per million (10 ppm) by weight and/or volume, among other amounts of silver particles 200 greater than or less than about 10 ppm by weight and/or volume that are possible, each of which is contemplated herein. In other embodiments, the total amount of silver particles 200 in a mixture 100 is about thirty-two parts per million (32 ppm) by weight and/or volume, among other amounts of silver particles 200 greater than or less than about 32 ppm by weight and/or volume that are possible, each of which is contemplated herein.

As the size of the silver particles 200 becomes smaller, a given concentration of the silver particles 200 will represent a larger quantity of particles 200. In addition, the total surface area for a given particle concentration of the silver particles 200 will increase. Therefore, particle size and/or range of particle size for the silver particles 200 may further characterize the mixture 100 of the various embodiments, which may include any suitable particle size(s) and/or range(s) of particle size(s) that is/are known or developed in the future capable of being infused or otherwise mixed with the silicone 102 to form the mixture 100.

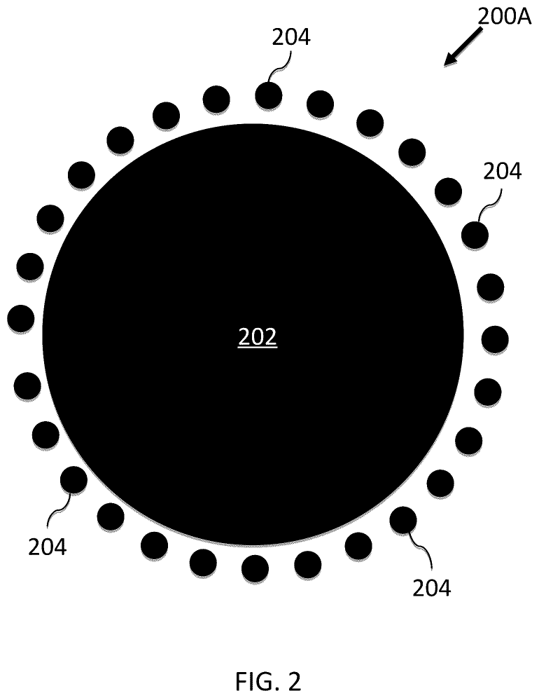

FIG. 2 is a diagram of one embodiment of a silver particle 200, which can also be referred to herein as, a nano-silver particle 200A. The nano-silver particle 200A may be characterized in terms of valence of the silver included therein. In the exemplary embodiment shown in FIG. 2, the nano-silver particle 200A includes a silver core 202 and a silver coating 204.

A silver core 202, in various embodiments, includes metallic silver (Ag(0)) to form a metallic silver core. In additional embodiments, the silver core 202 includes metallic nano-silver to form a metallic nano-silver core.

In various embodiments, the silver coating 204 comprises Ag(I), Ag(II), or Ag(III) in an ionic oxidation state. In some embodiments, the silver coating 204 includes silver oxide (e.g., AgO). That is, the nano-silver particle 200A comprises Ag(0) and AgO, where AgO is present as a coating on the silver core 202 (e.g., a metallic nano-silver core).

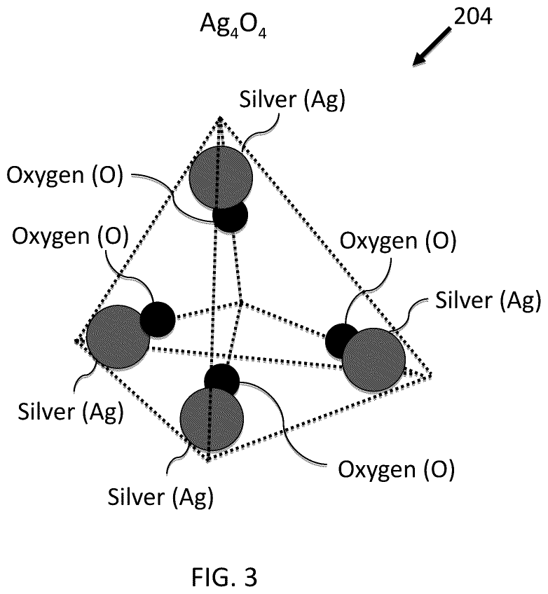

In other embodiments (see, FIG. 3), the silver coating 204 may be at least partially in the form of silver II oxide (Ag₄O₄). In a molecule of this material, two of the silver atoms are in the 1+ state (silver I) and the other two silver molecules are in the 3+ state (silver III). Under certain conditions, these molecules can give rise to silver atoms (Ag) in the 2+ (silver II) state attached to respective oxygen atoms (O). Thus, the present disclosure provides silver particles comprising a metallic silver core 202 and silver coating 204 of silver oxide, the silver oxide being selected from AgO and Ag₄O₄.

The purity of a nano-silver particle 200A may be any suitable percentage of silver purity that is capable of disinfecting a surface. In various embodiments, the percentage of silver purity of a nano-silver particle 200A is in the range of about 50% to about 99.9999%, among other percentages of silver purity that are greater than 99.9999% or less than 50% that are possible and contemplated herein. In certain embodiments, the percentage of silver purity of a nano-silver particle 200A is about 99.99%, among other percentages of silver purity that are greater than or less than about 99.99% that are possible and contemplated herein.

With reference to FIG. 4A, FIG. 4A is a block diagram of one embodiment of a silicone strip 400. The silicone strip 400, in various embodiments, is formed from a mixture 100A. That is, the silicone strip 400 is formed from a solid and/or semi-solid mixture 100A that includes an amount of silicone 102 infused with an amount of silver particles 200 and/or nano-silver particles 200A.

While referred to as a strip, the silicone strip 400 can also include silicone tape. That is, the silicone strip 400 can form a silicone tape infused with silver particles 200 and/or nano-silver particles 200A. As such, reference to a silicone strip 400 can also refer to silicone tape.

The silicone strip 400 may include any suitable size and/or dimensions (e.g., length, width, height, thickness, etc.) that is/are known or developed in the future for a target use and/or application of the silicone strip 400, which can include any suitable use/application of silicon, particles of silver, and/or silver that is/are known or developed in the future. In certain embodiments, the silicone strip 400 may include a consistency and/or texture similar to rubber, among other similar solid and/or semi-solid consistencies and/or textures that are possible, each of which is contemplated herein.

In additional or alternative embodiments, the silicone 102 in the silicone strip 400 includes medical grade silicone. The silicon strip 400, in further additional or alternative embodiments, is configured for application to the skin of a subject (e.g., human, animal, etc.). In certain embodiments, the silicone strip 400 is topically applied to the skin of the subject to treat a wound, burn, scar, and/or contracture thereon.

Referring to FIG. 4B, FIG. 4B is a diagram of one embodiment of a mixture 100B. The mixture 100B includes liquid, semi-liquid, and/or gel silicone 102 infused with silver particles 200 and/or nano-silver particles 200A. As such, the mixture 100B can include a liquid, semi-liquid, and/or gel state, form, consistency and/or texture.

The mixture 100B may include any suitable characteristic(s) (e.g., viscosity, thickness, color, etc.) that is/are known or developed in the future for a target use and/or application of the mixture 100B, which can include any suitable use/application of silicon, particles of silver, and/or silver that is/are known or developed in the future. That is, various embodiments of the mixture 100B can include liquid silicone 102 infused with silver particles 200 and/or nano-silver particles 200A, semi-liquid silicone 102 infused with silver particles 200 and/or nano-silver particles 200A, and/or a gel of silicone 102 infused with silver particles 200 and/or nano-silver particles 200A.

In various embodiments, the silicone 102 in the mixture 100B includes medical grade silicone. The mixture 100B, in additional or alternative embodiments, is configured for application to the skin of a subject (e.g., human, animal, etc.). In certain embodiments, the mixture 100B is topically applied to the skin of the subject to treat a wound, burn, scar, and/or contracture thereon.

With reference again to FIG. 1, the mixture 100 (including mixtures 100A and/or 100B) can be created and/or generated using any suitable technique, process, and/or method that is known or developed in the future capable of infusing an amount of silver particles 200 (including nano-silver particles 200A) with silicone 102. As discussed elsewhere herein, the mixture 100 may include a strip of silicone 102 (e.g., silicone strip 400), a silicone tape (e.g., silicone strip 400), a solid silicone 102 (e.g., silicone strip 400, mixture 100A, mixture 100B, etc.), a semi-solid silicone 102 (e.g., silicone strip 400, mixture 100A, mixture 100B, etc.), a liquid silicone 102 (e.g., mixture 100B), a semi-liquid silicone 102 (e.g., mixture 100B), and/or a gel of silicone 102 (e.g., mixture 100B) infused with an amount of silver particles 200 (and/or nano-silver particles 200A).

In certain embodiments, the silver particles 200 and/or silver particles 200A are infused with, added to, and/or mixed with one or more liquids and/or solutions to form a silver composition.

The silver composition may include any suitable liquid(s) and/or solution(s) that can facilitate, enable, and/or allow the silver particles 200 and/or silver particles 200A to be infused with silicone 102.

In various embodiments, the silver composition includes an amount of the silver particles 200 and/or silver particles 200A that, in whole or in part, comprise silver in an aqueous medium or solution. In various embodiments, the aqueous medium or solution includes water (H₂O) or other aqueous solution. In some embodiments, the water or aqueous solution includes pure water and/or purified water.

A silver composition comprising the silver particles 200 and/or silver particles 200A may be prepared using any suitable method and/or technique that is known or developed in the future. For example, the preparation of a composition comprising silver particles 200 and/or silver particles 200A may utilize an electrochemical cell comprising electrodes. One process includes: (a) placing a silver electrode in contact with a quantity of high purity water; (b) conveying electrical current through the silver electrode to thereby separate particles of silver from the silver electrode in a manner sufficient to cause production of suspended silver particles within the water; and (c) agitating the water during said production of suspended silver particles to thereby disperse the silver particles into a more uniform concentration within the water such that a higher quantity of silver particles can be produced per batch.

Another example technique for preparing a composition comprising silver particles 200 and/or silver particles 200A includes: (a) establishing an electrical circuit comprising a current source, and a first conductor electrically connected to the current source and a second conductor electrically connected to the current source in which the first conductor is spaced apart from the second conductor, and at least one of the conductors is made of elemental silver; (b) closing the circuit by placing the first conductor and the second conductor in communication with a fluidic resistor; (c) operating the current source to supply alternating current simultaneously to the first conductor and the second conductor such that voltage is increasing and decreasing within the first and second conductors in alternating tandem to thereby cause silver particles to separate from the first electrode and enter the fluidic resistor and become disposed in suspension within the fluidic resistor; and (d) selectively adjusting the electrodes by moving them toward the fluidic resistor to compensate for decrease in electrode length due to gradual separation of silver particles therefrom to thereby prevent arcing from occurring between the electrodes and said fluidic resistor.

In some embodiments, the silver composition comprises 0.1-10% of the nano-silver particles 200A by weight and/or volume including a maximum dimension that is less than about 1.0 micrometer (1000 nanometers (nm)), among other percentages and sizes that are possible, each of which contemplated herein. In certain embodiments, a silver composition comprises 0.001-1% of the nano-silver particles 200A including a dimension in the range of about .005 micrometers (5 nm) to about 0.01 micrometers (10 nm), among other percentages and sizes that are possible, each of which is contemplated herein.

In various embodiments, the total amount of nano-silver particles 200A in a silver composition is in the range of about 0.5 ppm to about 100 ppm by weight and/or volume, among other quantities of nano-silver particles 200A that are less than about 0.5 ppm and greater than about 100 ppm by weight and/or volume of particles of silver that are possible, each of which is contemplated herein. In some embodiments, the total amount of nano-silver particles 200A in a silver composition is about 10 ppm by weight and/or volume, among other amounts of nano-silver particles 200A greater than or less than about 10 ppm by weight and/or volume that are possible, each of which is contemplated herein. In other embodiments, the total amount of nano-silver particles 200A in a silver composition is about 32 ppm by weight and/or volume, among other amounts of nano-silver particles 200A greater than or less than about 32 ppm by weight and/or volume that are possible, each of which is contemplated herein.

In certain embodiments, a silver composition comprises 0.1-10% of the silver particles 200 including a minimum dimension greater than or equal to about 0.001 micrometers (1 nm), among other percentages and sizes that are possible, each of which is contemplated herein. Here, these silver particles 200 can be considered colloidal silver particles. In various embodiments, the silver solution includes about ten thousand parts per million (10,000 ppm) of the colloidal silver particles, among other amounts that are greater than or less than 10,000 ppm that are possible, each of which is contemplated herein.

In other embodiments, a silver composition comprises 0.1-10% of the silver particles 200 including a maximum dimension that is less than about 0.001 micrometers (1 nm), among other percentages and sizes that are possible, each of which is contemplated herein. Here, these silver particles 200 can be considered ionic silver particles or solution silver particles.

In still further embodiments, a silver composition comprises 0.1-10% of the silver particles 200 including a minimum dimension that is greater than about 1.0 micrometer (1000 nm), among other percentages and sizes that are possible and contemplated herein. Here, these silver particles 200 can be considered suspension silver particles.

In various embodiments, the silver particles 200 and/or silver solution form(s) a silver sol. A silver sol, in various embodiments, includes a stable dispersion of solid silver particles 200 homogeneously dispersed in a liquid. In certain embodiments, the silver sol includes a stable dispersion of solid silver particles 200 homogeneously dispersed in water (e.g., pure water) and/or an aqueous solution.

A silver solution including silver particles 200 and/or nano-silver particles 200A can be added to and/or mixed with an amount of silicone 102. As discussed elsewhere herein, the mixture 100 (including mixtures 100A and/or 100B) can be created and/or generated using any suitable technique, process, and/or method that is known or developed in the future capable of infusing an amount of silver particles 200 (including nano-silver particles 200A) with silicone 102.

The following is one embodiment of creating a mixture 100 (including mixtures 100A and/or 100B). The following embodiment is not intended to limit the scope and/or spirit of the various embodiments, but rather, is intended for ease in understanding the various embodiments.

To produce silicone, silicon is isolated from silica. For example, an amount of quartz sand is heated to a predetermined temperature (e.g., 1800° C.) to isolate silicon (e.g., pure silicon) from the quartz sand. Upon cooling, the silicon is ground to a silicon powder (e.g., a fine silicon powder).

The silicon powder is mixed with methyl chloride. Heating the mixture of silicon powder and methyl chloride causes a reaction that results in methyl chlorosilane, which includes dimethyldichlorosilane. The methyl chlorosilane is distilled (e.g., heated to a series of temperatures) to separate the dimethyldichlorosilane from the methyl chlorosilane.

A silver solution that includes water is added to the dimethyldichlorosilane, which causes a separation of hydrochloric acid and disilanol. The hydrochloric acid acts as a catalyst causing the disilanol to condense into polydimethylsiloxane including silver particles 200 and/or nano-silver particles 200A.

The polydimethylsiloxane including silver particles 200 and/or nano-silver particles 200A is then polymerized. The polydimethylsiloxane including silver particles 200 and/or nano-silver particles 200A can polymerized using any suitable technique, process and/or method that is known in the art or developed in the future. In various embodiments, the polydimethylsiloxane including silver particles 200 and/or nano-silver particles 200A is polymerized in accordance with one or more desired properties of a target use and/or application of the mixture 100 (e.g., mixture 100A, mixture 100B, silicone strip 400, and/or silicone tape, etc.).

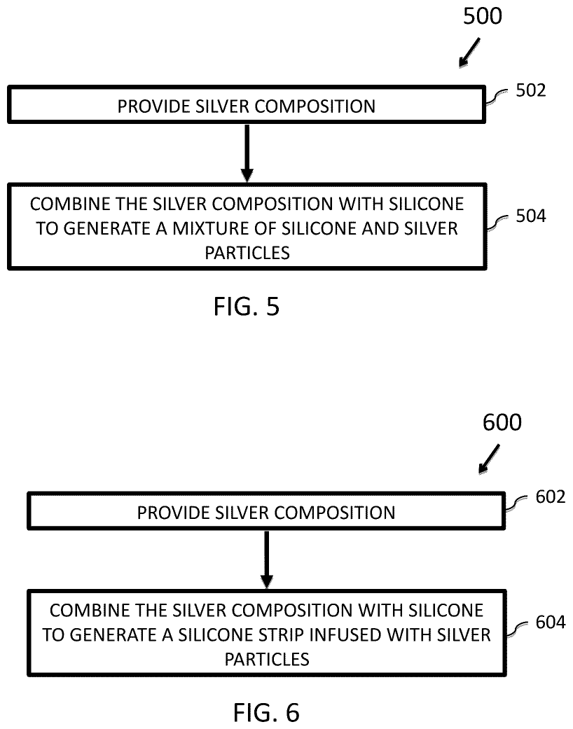

With reference to FIG. 5, FIG. 5 is a schematic flow diagram of one embodiment of a method 500 for generating a mixture 100, 100A, or 100B of silicone 102 infused with silver particles 200 or 200A. At least in the illustrated embodiment, the method 500 begins by providing a silver composition (block 502).

The silver composition may include any suitable amount of silver particles 200, 200A that can produce a mixture 100, 100A, or 100B including silicone 102 infused with a desired amount and/or concentration of silver particles 200 or 200A. In various embodiments, the silver composition includes an amount of silver particles 200, 200A (e.g., Ag particles) in the range of about 0.5 ppm to about 100 ppm, as discussed elsewhere herein. In some embodiments, the silver composition includes about 10 ppm of silver particles 200, 200A, as discussed elsewhere herein. In other embodiments, the silver composition includes about 32 ppm of silver particles 200, 200A, as discussed elsewhere herein.

The method 500 further includes combining the silver composition with silicone 102 to generate a mixture 100, 100A, 100B of silicone 102 infused with silver particles 200, 200A (block 504). The silver composition and silicone 102 may be combined using any suitable method, process, and/or technique that is known, developed in the future, and/or discussed herein that is capable of producing a mixture 100, 100A, 100B of silicone 102 infused with silver particles 200, 200A.

The silicone 102 may include any of the embodiments of the silicone 102 discussed elsewhere herein. In some embodiments, the silicone 102 includes a medical grade silicone.

Further, the mixture 100, 100A, 100B may include any suitable amount and/or concentration of silver particles 200, 200A for a particular/desired/target use and/or application of the mixture 100, 100A, 100B. In various embodiments, the mixture 100, 100A, 100B includes an amount of silver particles 200, 200A (e.g., Ag particles) in the range of about 0.5 ppm to about 100 ppm, as discussed elsewhere herein. In some embodiments, the mixture 100, 100A, 100B includes about 10 ppm of silver particles 200, 200A, as discussed elsewhere herein. In other embodiments, the mixture 100, 100A, 100B includes about 32 ppm of silver particles 200, 200A, as discussed elsewhere herein.

Referring to FIG. 6, FIG. 6 is a schematic flow diagram of one embodiment of a method 600 for generating a silicone strip (e.g., silicone strip 400) infused with silver particles 200, 200A. At least in the illustrated embodiment, the method 600 begins by providing a silver composition (block 602).

The silver composition may include any suitable amount of silver particles 200, 200A that can produce a silicone strip 400 including silicone 102 infused with a desired amount and/or concentration of silver particles 200 or 200A. In various embodiments, the silver composition includes an amount of silver particles 200, 200A (e.g., Ag particles) in the range of about 0.5 ppm to about 100 ppm, as discussed elsewhere herein. In some embodiments, the silver composition includes about 10 ppm of silver particles 200, 200A, as discussed elsewhere herein. In other embodiments, the silver composition includes about 32 ppm of silver particles 200, 200A, as discussed elsewhere herein.

The method 600 further includes combining the silver composition with silicone 102 to generate a silicone strip 400 that includes silicone 102 infused with silver particles 200, 200A (block 604). The silver composition and silicone 102 may be combined using any suitable method, process, and/or technique that is known, developed in the future, and/or discussed herein that is capable of producing a silicone strip 400 that includes silicone 102 infused with silver particles 200, 200A.

The silicone 102 may include any of the embodiments of the silicone 102 discussed elsewhere herein. In some embodiments, the silicone 102 includes a medical grade silicone.

Further, the silicone strip 400 may include any suitable amount and/or concentration of silver particles 200, 200A for a particular/desired/target use and/or application of the silicone strip 400. In various embodiments, the silicone strip 400 includes an amount of silver particles 200, 200A (e.g., Ag particles) in the range of about 0.5 ppm to about 100 ppm, as discussed elsewhere herein. In some embodiments, the silicone strip 400 includes about 10 ppm of silver particles 200, 200A, as discussed elsewhere herein. In other embodiments, the silicone strip 400 includes about 32 ppm of silver particles 200, 200A, as discussed elsewhere herein.

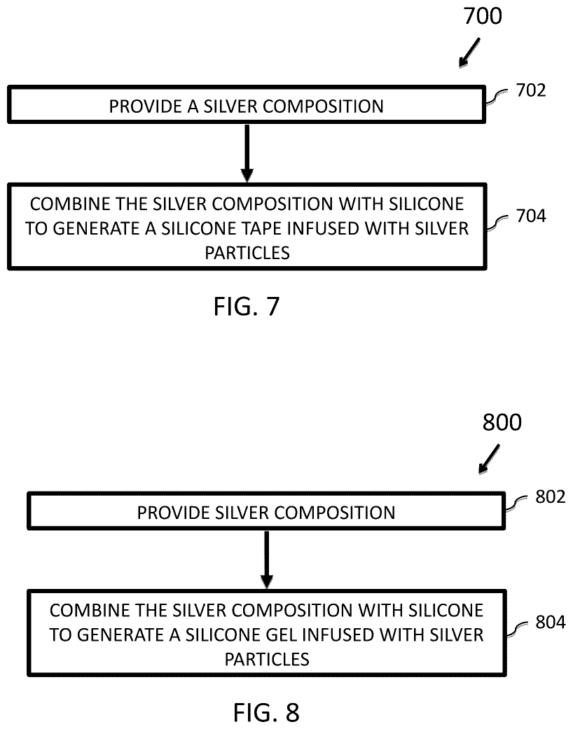

With reference to FIG. 7, FIG. 7 is a schematic flow diagram of one embodiment of a method 700 for generating a silicone tape infused with silver particles 200, 200A. At least in the illustrated embodiment, the method 700 begins by providing a silver composition (block 702).

The silver composition may include any suitable amount of silver particles 200, 200A that can produce a silicone tape including silicone 102 infused with a desired amount and/or concentration of silver particles 200 or 200A. In various embodiments, the silver composition includes an amount of silver particles 200, 200A (e.g., Ag particles) in the range of about 0.5 ppm to about 100 ppm, as discussed elsewhere herein. In some embodiments, the silver composition includes about 10 ppm of silver particles 200, 200A, as discussed elsewhere herein. In other embodiments, the silver composition includes about 32 ppm of silver particles 200, 200A, as discussed elsewhere herein.

The method 700 further includes combining the silver composition with silicone 102 to generate a silicone tape including silicone 102 infused with silver particles 200, 200A (block 704). The silver composition and silicone 102 may be combined using any suitable method, process, and/or technique that is known, developed in the future, and/or discussed herein that is capable of producing a silicone tape including silicone 102 infused with silver particles 200, 200A.

The silicone 102 may include any of the embodiments of the silicone 102 discussed elsewhere herein. In some embodiments, the silicone 102 includes a medical grade silicone.

Further, the silicone tape may include any suitable amount and/or concentration of silver particles 200, 200A for a particular/desired/target use and/or application of the silicone tape. In various embodiments, the silicone tape includes an amount of silver particles 200, 200A (e.g., Ag particles) in the range of about 0.5 ppm to about 100 ppm, as discussed elsewhere herein. In some embodiments, the silicone tape includes about 10 ppm of silver particles 200, 200A, as discussed elsewhere herein. In other embodiments, the silicone tape includes about 32 ppm of silver particles 200, 200A, as discussed elsewhere herein.

Referring to FIG. 8, FIG. 8 is a schematic flow diagram of one embodiment of a method 800 for generating a silicone gel infused with silver particles. At least in the illustrated embodiment, the method 800 begins by providing a silver composition (block 802).

The silver composition may include any suitable amount of silver particles 200, 200A that can produce a silicone gel including silicone 102 infused with a desired amount and/or concentration of silver particles 200 or 200A. In various embodiments, the silver composition includes an amount of silver particles 200, 200A (e.g., Ag particles) in the range of about 0.5 ppm to about 100 ppm, as discussed elsewhere herein. In some embodiments, the silver composition includes about 10 ppm of silver particles 200, 200A, as discussed elsewhere herein. In other embodiments, the silver composition includes about 32 ppm of silver particles 200, 200A, as discussed elsewhere herein.

The method 800 further includes combining the silver composition with silicone 102 to generate a silicone gel including silicone 102 infused with silver particles 200, 200A (block 804). The silver composition and silicone 102 may be combined using any suitable method, process, and/or technique that is known, developed in the future, and/or discussed herein that is capable of producing a silicone gel including silicone 102 infused with silver particles 200, 200A.

The silicone 102 may include any of the embodiments of the silicone 102 discussed elsewhere herein. In some embodiments, the silicone 102 includes a medical grade silicone.

Further, the silicone gel may include any suitable amount and/or concentration of silver particles 200, 200A for a particular/desired/target use and/or application of the silicone gel. In various embodiments, the silicone gel includes an amount of silver particles 200, 200A (e.g., Ag particles) in the range of about 0.5 ppm to about 100 ppm, as discussed elsewhere herein. In some embodiments, the silicone gel includes about 10 ppm of silver particles 200, 200A, as discussed elsewhere herein. In other embodiments, the silicone gel includes about 32 ppm of silver particles 200, 200A, as discussed elsewhere herein.

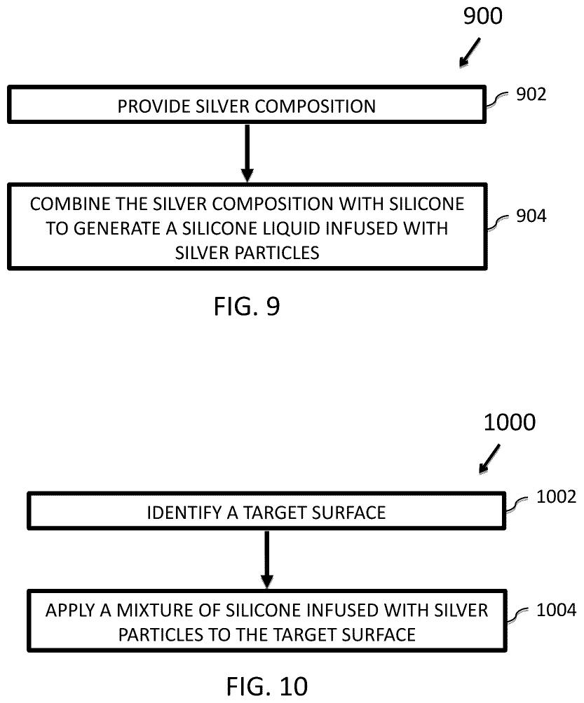

With reference to FIG. 9, FIG. 9 is a schematic flow diagram of one embodiment of a method 900 for generating a silicone liquid infused with silver particles 200, 200A. At least in the illustrated embodiment, the method 700 begins by providing a silver composition (block 902).

The silver composition may include any suitable amount of silver particles 200, 200A that can produce a silicone liquid including silicone 102 infused with a desired amount and/or concentration of silver particles 200 or 200A. In various embodiments, the silver composition includes an amount of silver particles 200, 200A (e.g., Ag particles) in the range of about 0.5 ppm to about 100 ppm, as discussed elsewhere herein. In some embodiments, the silver composition includes about 10 ppm of silver particles 200, 200A, as discussed elsewhere herein. In other embodiments, the silver composition includes about 32 ppm of silver particles 200, 200A, as discussed elsewhere herein.

The method 900 further includes combining the silver composition with silicone 102 to generate a silicone liquid including silicone 102 infused with silver particles 200, 200A (block 904). The silver composition and silicone 102 may be combined using any suitable method, process, and/or technique that is known, developed in the future, and/or discussed herein that is capable of producing a silicone liquid including silicone 102 infused with silver particles 200, 200A.

The silicone 102 may include any of the embodiments of the silicone 102 discussed elsewhere herein. In some embodiments, the silicone 102 includes a medical grade silicone.

Further, the silicone liquid may include any suitable amount and/or concentration of silver particles 200, 200A for a particular/desired/target use and/or application of the silicone liquid. In various embodiments, the silicone liquid includes an amount of silver particles 200, 200A (e.g., Ag particles) in the range of about 0.5 ppm to about 100 ppm, as discussed elsewhere herein. In some embodiments, the silicone liquid includes about 10 ppm of silver particles 200, 200A, as discussed elsewhere herein. In other embodiments, the silicone liquid includes about 32 ppm of silver particles 200, 200A, as discussed elsewhere herein.

Referring to FIG. 10, FIG. 10 is a schematic flow diagram of one embodiment of a method 1000 for treating a target surface with a mixture 100, 100A, 100B of silicone 102 infused with silver particles 200, 200A. At least in the illustrated embodiment, the method 1000 begins by identifying a target surface (block 1002).

The target surface may include any suitable surface and/or type of surface that can benefit from the application of a mixture 100, 100A, 100B of silicone 102 infused with silver particles 200, 200A applied thereon. In various embodiments, the target surface includes the skin of a subject (e.g., human, animal, etc.).

The method 1000 further includes applying the mixture 100, 100A, 100B of silicone 102 infused with silver particles 200, 200A to the target surface (block 1004). The mixture 100, 100A, 100B of silicone 102 infused with silver particles 200, 200A may be applied using any suitable method, process, and/or technique that is known or developed in the future capable of applying the mixture 100, 100A, 100B of silicone 102 infused with silver particles 200, 200A to, on, and/or over a desired and/or target surface.

Further, the mixture 100, 100A, 100B of silicone 102 infused with silver particles 200, 200A may include any suitable amount and/or concentration of silver particles 200, 200A for a particular/desired/target use and/or application of the mixture 100, 100A, 100B. In various embodiments, the mixture 100, 100A, 100B includes an amount of silver particles 200, 200A (e.g., Ag particles) in the range of about 0.5 ppm to about 100 ppm, as discussed elsewhere herein. In some embodiments, the mixture 100, 100A, 100B includes about 10 ppm of silver particles 200, 200A, as discussed elsewhere herein. In other embodiments, the mixture 100, 100A, 100B includes about 32 ppm of silver particles 200, 200A, as discussed elsewhere herein.

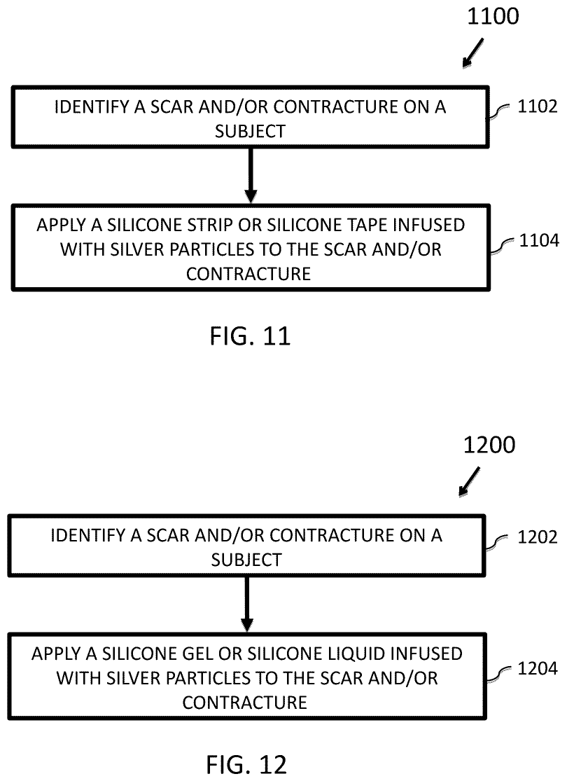

With reference to FIG. 11, FIG. 11 is a schematic flow diagram of one embodiment of a method 1100 for treating a scar and/or with a silicone strip 400 or silicone tape infused with silver particles 200, 200A. At least in the illustrated embodiment, the method 1100 begins by identifying a scar and/or contracture (block 1102), which can be located on the skin of a subject (e.g., human, animal, etc.).

The method 1100 further includes applying the silicone strip 400 or silicone tape to the scar and/or contracture (block 1104). The silicone strip 400 or silicone tape including silicone 102 infused with silver particles 200, 200A may be applied using any suitable method, process, and/or technique that is known or developed in the future capable of applying the silicone strip 400 or silicone tape to, on, and/or over a desired and/or target scar and/or contracture.

Further, the silicone strip 400 or silicone tape including silicone 102 infused with silver particles 200, 200A may include any suitable amount and/or concentration of silver particles 200, 200A for a particular/desired/target use and/or application of the silicone strip 400 or silicone tape to a scar and/or contracture. In various embodiments, the silicone strip 400 or silicone tape includes an amount of silver particles 200, 200A (e.g., Ag particles) in the range of about 0.5 ppm to about 100 ppm, as discussed elsewhere herein. In some embodiments, the silicone strip 400 or silicone tape includes about 10 ppm of silver particles 200, 200A, as discussed elsewhere herein. In other embodiments, the silicone strip 400 or silicone tape includes about 32 ppm of silver particles 200, 200A, as discussed elsewhere herein.

Referring to FIG. 12, FIG. 12 is a schematic flow diagram of one embodiment of a method 1200 for treating a scar and/or with a silicone gel or silicone liquid infused with silver particles 200, 200A. At least in the illustrated embodiment, the method 1200 begins by identifying a scar and/or contracture (block 1202), which can be located on the skin of a subject (e.g., human, animal, etc.).

The method 1200 further includes applying the silicone gel or silicone liquid to the scar and/or contracture (block 1204). The silicone gel or silicone liquid including silicone 102 infused with silver particles 200, 200A may be applied using any suitable method, process, and/or technique that is known or developed in the future capable of applying the silicone gel or silicone liquid to, on, and/or over a desired and/or target scar and/or contracture.

Further, the silicone gel or silicone liquid including silicone 102 infused with silver particles 200, 200A may include any suitable amount and/or concentration of silver particles 200, 200A for a particular/desired/target use and/or application of the silicone gel or silicone liquid to a scar and/or contracture. In various embodiments, the silicone gel or silicone liquid includes an amount of silver particles 200, 200A (e.g., Ag particles) in the range of about 0.5 ppm to about 100 ppm, as discussed elsewhere herein. In some embodiments, the silicone gel or silicone liquid includes about 10 ppm of silver particles 200, 200A, as discussed elsewhere herein. In other embodiments, the silicone gel or silicone liquid includes about 32 ppm of silver particles 200, 200A, as discussed elsewhere herein.

Reference throughout this specification to one embodiment, an embodiment, or similar language means that a particular feature, structure, or characteristic described in connection with the embodiment is included in at least one embodiment. Thus, appearances of the phrases in one embodiment, in an embodiment, and similar language throughout this specification may, but do not necessarily, all refer to the same embodiment, but mean one or more but not all embodiments unless expressly specified otherwise. The terms including, comprising, having, and variations thereof mean including but not limited to, unless expressly specified otherwise. An enumerated listing of items does not imply that any or all of the items are mutually exclusive, unless expressly specified otherwise. The terms, “a,” “an,” and “the,” also refer to one or more unless expressly specified otherwise.

In addition, as used herein, the term, “set,” can mean one or more, unless expressly specified otherwise. The term, “sets,” can mean multiples of or a plurality of one or mores, ones or more, and/or ones or mores consistent with set theory, unless expressly specified otherwise.

Furthermore, the described features, structures, or characteristics of the embodiments may be combined in any suitable manner. In the above description, numerous specific details are provided, such as examples of programming, software modules, user selections, network transactions, database queries, database structures, hardware modules, hardware circuits, hardware chips, etc., to provide a thorough understanding of embodiments. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

It should also be noted that, in some alternative implementations, the functions noted in the method blocks may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment.

The description of elements in each figure may refer to elements of proceeding figures. Like numbers refer to like elements in all figures, including alternate embodiments of like elements.

The various disclosed embodiments may be practiced in other specific forms. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
silicone forming a silicone strip; and
an amount of silver (Ag) particles infused in the silicone strip.

2. The mixture of claim 1, wherein the Ag particles comprise nano-silver particles.

3. The mixture of claim 2, wherein the mixture comprises 0.5 ppm to 100 ppm by weight of the Ag particles.

4. The mixture of claim 2, wherein the mixture comprises 10 ppm by weight of the Ag particles.

5. The mixture of claim 2, wherein the mixture comprises 32 ppm by weight of the Ag particles.

6. The mixture of claim 2, wherein the nano-silver particles include a size less than or equal to one micrometer (1 μm).

7. The mixture of claim 1, wherein the Ag particles comprise colloidal silver particles.

8. A mixture, comprising:
silicone; and
an amount of silver (Ag) particles infused in the silicone,
wherein the mixture includes one of a liquid or a gel.

9. The mixture of claim 8, wherein the Ag particles comprise nano-silver particles.

10. The mixture of claim 9, wherein the mixture comprises 0.5 ppm to 100 ppm by weight of the Ag particles.

11. The mixture of claim 9, wherein the mixture comprises 10 ppm by weight of the Ag particles.

12. The mixture of claim 9, wherein the mixture comprises 32 ppm by weight of the Ag particles.

13. The mixture of claim 9, wherein the nano-silver particles include a size less than or equal to one micrometer (1 μm).

14. The mixture of claim 8, wherein the Ag particles comprise colloidal silver particles.

15. A method, comprising:
identifying a target surface; and
applying a mixture to the target surface,
wherein the mixture includes silicone and an amount of silver (Ag) particles infused in the silicone.

16. The method of claim 15, wherein:
the mixture forms a silicone strip infused with the amount of the silver particles; and
the target surface includes skin.

17. The method of claim 15, wherein the Ag particles comprise nano-silver particles.

18. The method of claim 17, wherein the mixture comprises 0.5 ppm to 100 ppm by weight of the Ag particles.

19. The method of claim 17, wherein the nano-silver particles include a size less than or equal to one micrometer (1 μm).

20. The method of claim 15, wherein the Ag particles comprise colloidal silver particles.
